(19) United States
(12) Patent Application Publication
(10) Pub. No.: US 2024/0366551 A1
(43) Pub. Date: Nov. 7, 2024

(54) MIXTURES AND COMPOSITIONS COMPRISING SULFORAPHANE AND GLYCINE

(71) Applicants: SOCIETE DES PRODUITS NESTLE S.A., Vevey (CH); NORTHEASTERN UNIVERSITY, Boston, MA (US)

(72) Inventors: STEPHANIE BLUM-SPERISEN, Pully (CH); PHILIPP GUT, Geneve (CH); SEBASTIEN HERZIG, Morges (CH); JONATHAN THEVENET, Publier (FR); ALBERT-LASZLO BARABASI, Brookline, MA (US); ITALO FARIA DO VALLE, Natick, MA (US); PETER RUPPERT, Chestnut Hill, MA (US); FARZANEH NASIRIAN, Malden, MA (US)

(21) Appl. No.: 18/683,326

(22) PCT Filed: Aug. 16, 2022
(86) PCT No.: PCT/EP2022/072870

§ 371 Date: Feb. 13, 2024

Related U.S. Application Data
(60) Provisional application No. 63/234,436, filed on Aug. 18, 2021.

(51) Int. Cl. A61K 31/26; A61K 31/198; A61P 39/06

(52) U.S. Cl. CPC A61K 31/26; A61K 31/198; A61P 39/06

(57) ABSTRACT

The present disclosure generally relates to mixtures and compositions comprising sulforaphane, or analogue thereof, and at least one amino acid, or analogue thereof. In particular, the disclosure relates the use of the mixtures and compositions for treating or preventing oxidative stress.

### TECHNICAL FIELD

The present disclosure generally relates to mixtures and compositions comprising sulforaphane, or analogue thereof, and glycine, or analogue thereof. In particular, the disclosure relates to the use of the mixtures and compositions for treating or preventing oxidative stress.

### BACKGROUND

Oxidative stress is a phenomenon in which an imbalance exists between production and accumulation of oxygen reactive species (ROS) in cells and tissues and the ability of a biological system to detoxify these reactive products. Disturbances in the normal redox state of cells can cause toxic effects through the production of peroxides and free radicals that damage cell components including proteins, cell membranes, lipids, and DNA, ultimately resulting in cell death.

Free radicals and oxidants have been linked as contributors to many diseases and conditions, including: cancer (e.g. via DNA damage¹⁻³), cardiovascular disease⁴⁻⁶, neurological disease⁷⁻⁹, respiratory disease¹⁰⁻¹², rheumatoid arthritis^{2,13,14}, liver¹⁵ and kidney diseases¹⁶, inflammation¹⁷⁻¹⁹, diabetes^{20,21,} metabolism disorders (lipid and glucose)²²⁻²⁴ and aging^{25,26}.

In human-health perspective, it is impossible to completely avoid free radical exposure and oxidative stress. However, reduction of oxidative stress is possible by dietary supplementation with antioxidants. Vitamins C and E, flavonoids (a class of polyphenolic compounds) and carotenoids are examples of antioxidant compounds that have been studied comprehensively with respect to benefits to human health. Typically, health practitioners recommend consuming multiple servings per day of fruits and vegetables which are natural sources of such antioxidants. Consequently, the identification of other natural antioxidant compounds has been the focus of intensified research that seeks to further improve human health by combatting causative agents of oxidative stress.

Sulforaphane is a naturally occurring organosulfur compound (an isothiocyanate) found in vegetables of the family Brassicaceae. This family includes cauliflower, cabbage, kale, garden cress, bok choy, and Brussels sprouts, but most notably broccoli in which sulforaphane exists at high levels^{27,28}, Sulforaphane has a chemical structure 4-methylsulfinylbutyl isothiocyanate or 1-isothiocyanate-4-methylsulfinylbutane and is a phytochemical that occurs in plants in the form of biological inactive precursor glucoraphanin. Glucoraphanin belongs to the group of phytochemicals called glucosinolates that have a sugar component built in their structure (most often d-glucose) and are rapidly converted to isothiocyanate form by the enzyme called myrosinase²⁹. The process of conversion takes place after a disruption of plant tissues (e.g. by biting, chewing, slicing etc), when the enzyme myrosinase is released. Destruction of myrosinase during meal preparation by cooking or microwave treatment can significantly reduce isothiocyanate bioavailability, albeit that an alternative source of isothiocyanate following ingestion of cooked vegetables can be by degradation of glucosinolates by the intestinal microflora. Degradation by the microflora is, in any event, not efficient and can be variable^{30,31}.

Studies with sulforaphane in extracted or purified form have shown that the compound can be beneficial to humans in the context of combatting oxidative stress (as both an antioxidant³² and a chemoprotectant³³). Further investigation suggests that sulforaphane is a potent activator of transcription factor Nrf2, which in turn is a potent activator of cellular defences against oxidative stress³⁴.

It would therefore be desirable to provide compositions in which the beneficial activity of sulforaphane in combatting oxidative stress is enhanced.

Discussion or mention of any piece of prior art in this specification is not to be taken as an admission that the prior art is part of the common general knowledge of the skilled addressee of the specification.

### SUMMARY

This summary is provided to introduce a selection of concepts in a simplified form that are further described below in the Detailed Description. This summary is not intended to identify key features of the claimed subject matter, nor is it intended to be used as an aid in determining the scope of the claimed subject matter.

The present disclosure is predicated on the finding that the combination of sulforaphane, or analogue thereof, and glycine or analogue thereof may provide enhanced treatment or prevention of oxidative stress when compared to individual components applied separately.

According to one aspect of the disclosure there is provided a mixture comprising sulforaphane, or analogue thereof, and glycine, or analogue thereof.

In embodiments, the analogue of sulforaphane is selected from the group consisting of:
6-isothiocyanato-2-hexanone,
exo-2-acetyl-6-isothiocyanatonorbornane,
exo-2-isothiocyanato-6-methylsulfonylnorbornane,
6-isothiocyanato-2-hexanol,
1-isothiocyanato-4-dimethylphosphonylbutane,
exo-2-(1′-hydroxyethyl)-5-isothiocyanatonorborane,
exo-2-acetyl-5-isothiocyanoatonorbomane,
1-isothiocyanato-5-methylsulfonylpentane,
cis-or trans-3-(methylsulfonyl) cyclohexylmethylisothiocyanate, and combinations thereof.

In embodiments, the mixture is for use in the treatment or prevention of a disease or condition associated with oxidative stress, preferably at least one disease or condition selected from the group consisting of:
cancer,
cardiovascular disease,
neurological disease,
respiratory disease,
rheumatoid arthritis,
liver disease,
kidney disease,
inflammation,
diabetes,
metabolism disorders, and
aging.

In embodiments of the mixture of the present disclosure, sulforaphane, or analogue thereof, and glycine, or analogue thereof, are formulated for administration to a subject in need simultaneously or sequentially.

In embodiments of the mixture of the present disclosure, the molar ratio

of sulforaphane, or analogue thereof, to glycine, or analogue thereof, is between about 1:1000 to about 1000:1, preferably between about 1:800 to about 800:1, most preferably about 1:500.

In another aspect, the present disclosure provides a composition comprising:
sulforaphane, or analogue thereof,
glycine, or analogue thereof,
and optionally at least one additional ingredient.

In embodiments, the at least one ingredient is selected from the group consisting of:
lipids,
proteins,
carbohydrates,
pre-biotics,
pro-biotics,
essential fatty acids,
nucleotides,
nucleosides,
vitamins, and
minerals.

In yet another aspect, there is provided a method of manufacturing compositions of the present disclosure. In embodiments, the method comprises admixing the sulforaphane, or analogue thereof, with glycine, or analogue thereof and optionally the at least one additional ingredient.

In yet another aspect, the present disclosure provides methods of treating or preventing a disease or condition associated with oxidative stress.

Other embodiments of nutritional compositions according to the present disclosure will be evident from the following detailed description.

### DESCRIPTION OF FIGURES

FIG. 1. Shows survival of Zebrafish larvae in vitro in the absence and presence of hydrogen peroxide and other additives.

### DETAILED DESCRIPTION

### Definitions and Statements Regarding Interpretation

The terms “a,” “an,” “the” and similar referents used in the context of describing embodiments (especially in the context of the following claims) are to be construed to cover both the singular and the plural, unless otherwise indicated herein or clearly contradicted by context.

All methods and processes described herein can be performed in any suitable order unless otherwise indicated herein or otherwise clearly contradicted by context.

The use of any and all examples, or exemplary language (e.g., “such as”) provided herein is intended merely to illuminate and does not pose a limitation on the scope of the claims. No language in the specification should be construed as indicating any non-claimed element essential.

Recitation of ranges of values herein is merely intended to serve as a shorthand method of referring individually to each separate value falling within the range. Unless otherwise indicated herein, each individual value is incorporated into the specification as if it were individually recited herein. For example, if a range is from about 1 to about 50, it is deemed to include, for example, 1, 7, 34, 46.1, 23.7, or any other value or range within the range.

Unless noted otherwise, all percentages in the specification refer to weight percent, where applicable.

The terms “comprise”, “comprises”, “comprised” or “comprising”, “including” or “having” and the like in the present specification and claims are used in an inclusive sense, that is to specify the presence of the stated features but not preclude the presence of additional or further features.

Specific embodiments disclosed herein may be further limited in the claims using consisting of or consisting essentially of language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments so claimed are inherently or expressly described and enabled herein.

Unless defined otherwise, all technical and scientific terms have and should be given the same meaning as commonly understood by one of ordinary skill in the art to which this disclosure relates.

“Pharmaceutical composition”, “nutraceutical composition” or “composition” means a product manufactured to comprise one or more active ingredients, and may include one or more inert ingredients, as well as any product which results, directly or indirectly, from combination, complexation or aggregation of any two or more of the ingredients, or from dissociation of one or more of the ingredients, or from other types of reactions or interactions of one or more of the ingredients. Accordingly, the compositions of the present disclosure encompass any composition made by admixing a compound, substance or ingredient of the present disclosure and optionally a pharmaceutically or nutraceutically acceptable excipient (pharmaceutically acceptable carrier).

It is to be understood that pharmaceutically or nutraceutically acceptable derivatives of bioactive substances are included within the scope of the present disclosure.

The term “pharmaceutically or nutraceutically acceptable derivatives” includes, but is not limited to, pharmaceutically or nutraceutically acceptable salts, esters, salts of such esters, ethers, or any other derivative including prodrugs and metabolites, which upon administration to a subject (e.g. patient, human or animal) in need is capable of providing, directly or indirectly, a bioactive substance as otherwise described herein.

As used herein, the term “pharmaceutically or nutraceutically acceptable salt” refers to those salts which are, within the scope of sound medical judgment, suitable for use in contact with the tissues of humans and lower animal without undue toxicity, irritation, allergic response and the like, and are commensurate with a reasonable benefit/risk ratio.

The term “sulforaphane analogue” is used herein to refer to naturally occurring analogues of sulforaphane, such as, for example, sulforaphene, Erucin (sulforaphane with the sulphur not oxidized, i.e. methyl sulphide group) and Erysolin (sulforaphane with the sulphur overoxidized, i.e. methyl sulfone group).

An “amino acid analogue” (or “non-standard amino acid”) is meant to include all amino acid-like compounds that are similar in structure and/or overall shape to one or more of the twenty L-amino acids commonly found in naturally occurring proteins (Ala or A, Cys or C, Asp or D, Glu or E, Phe or F, Gly or G, His or H, Ile or I, Lys or K, Leu or L, Met or M, Asn or N, Pro or P, Gln or Q, Arg or R, Ser or S, Thr or T, Val or V, Trp or W, Tyr or Y, as defined and listed in WIPO Standard ST.25 (2009), Annex C, Appendix 2, Table 3). An amino acid analogue can include a natural amino acid that comprises a modified side chain or backbone. A modification may include, without limitation, substitution of an atom (such as N) for a related atom (such as S), addition of a group (such as methyl, or hydroxyl group, for example) or an atom (such as Cl or Br, for example), deletion of a group, substitution of a covalent bond (single bond for double bond, for example), or combinations thereof (see also WIPO Standard ST.25 (2009), Annex C, Appendix 2, Table 4, for example). As used herein, reference to “amino acid” with respect to compositions and methods for combating oxidative stress includes amino acid analogues.

An “analogue” of a substance in other contexts includes a functionally equivalent substance. Functional equivalence may be achieved by modifying the structure of the original substance using chemical or recombinant techniques, or by identifying a substance having a different structure having the same or similar function as the original substance.

The term “mixture” refers to a combination of components (compounds, substances or ingredients and the like), where components of the mixture are prepared individually, and where the mixture is achieved by admixing components before or during use. To this extent, a mixture may be achieved by admixing components before administration of the mixture to a subject in need. In embodiments, a mixture may be achieved by simultaneous or sequential administration of components. This therefore includes mixtures achieved within a subject in need.

A compound, substance or ingredient used in producing mixtures or compositions of the present disclosure may be derived from any conceivable source. This may include animal, vegetable, mineral or synthetic ('man-made') sources.

The term “isolated” or “purified” refers to a material that is removed from its original environment (e.g. the natural environment, if it is naturally occurring). For example, the material is said to be “purified” when it is present in a particular composition or mixture in a higher concentration than exists in a naturally occurring or wild type organism or in combination with components not normally present upon expression from a naturally occurring or wild type organism. For example, a naturally-occurring protein/polypeptide present in a living organism is not isolated, but the same protein/polypeptide, separated from some or all of the coexisting materials in the natural system, is isolated. Such proteins/polypeptides could, for example, be part of a composition, and still be isolated in that such a composition is not part of the natural environment of the proteins/polypeptides.

The terms “administration concurrently” or “co-administering” and the like refer to the administration of a single composition containing two or more actives, or the administration of each active as separate compositions and/or delivered by separate routes either contemporaneously or simultaneously or sequentially within a short enough period of time that the effective result is equivalent to that obtained when all such actives are administered as a single composition. By “simultaneously” is meant that the active agents are administered at substantially the same time, and preferably together in the same formulation.

As used herein, “treat”, “treating” or “treatment” of a disease or disorder means accomplishing one or more of the following: (a) reducing the severity and/or duration of the disorder; (b) limiting or preventing development of symptoms characteristic of the disorder(s) being treated; (c) inhibiting worsening of symptoms characteristic of the disorder(s) being treated; (d) limiting or preventing recurrence of the disorder(s) in patients that have previously had the disorder(s); and (e) limiting or preventing recurrence of symptoms in patients that were previously symptomatic for the disorder(s). As used herein, “prevent”, “preventing”, “prevention”, or “prophylaxis” of a disease or disorder means preventing that a disease or disorder occurs in subject.

The terms “effective amount” or “therapeutic amount” are intended to mean that amount of a substance that will elicit the biological or medical response of a tissue, a system, animal or human that is being sought by a researcher, veterinarian, medical doctor or other clinician. The term “prophylactically effective amount” is intended to mean that amount of a substance that will prevent or reduce the risk of occurrence of the biological or medical event that is sought to be prevented in a tissue, a system, animal or human by a researcher, veterinarian, medical doctor or other clinician.

As used herein, the expression “is for administration” and “is to be administered” have the same meaning as “is prepared to be administered”. In other words, the statement that an active compound “is for administration” has to be understood in that said active compound has been formulated and made up into doses so that said active compound is in a state capable of exerting its therapeutic activity.

### Mixtures According to the Disclosure

The present disclosure is directed to mixtures for treating or preventing oxidative stress.

In one aspect, the present disclosure provides mixtures comprising sulforaphane, or analogue thereof, and glycine, or analogue thereof.

In embodiments, the sulforaphane is 4-methylsulfinylbutyl isothiocyanate or 1-isothiocyanato-4-(methylsulfinyl) butane.

In other embodiments, sulforaphane analogues useful for preparing the present mixtures include one or more of: 6-isothiocyanato-2-hexanone, exo-2-acetyl-6-isothiocyanatonorbornane, exo-2-isothiocyanato-6-methylsulfonylnorbornane, 6-isothiocyanato-2-hexanol, 1-isothiocyanato-4-dimethylphosphonylbutane, exo-2-(1′-hydroxyethyl)-5-isothiocyanatonorborane, exo-2-acetyl-5-isothiocyanoatonorbomane, 1-isothiocyanato-5-methylsulfonylpentane, and cis-or trans-3-(methylsulfonyl)cyclohexylmethylisothiocyanate, or mixtures thereof.

Preferably, sulforaphane and/or sulforaphane analogues of the present disclosure may be from any suitable natural source. For example, sulforaphane and/or its analogues may be extracted from broccoli, Brussel sprouts, cabbage, cauliflower, bok choy, kale, collards, Chinese broccoli, broccoli raab, kohlrabi, mustard, turnip, radish, arugula, watercress or other natural sources. Therefore, in embodiments, the natural source is selected from one or more of these natural vegetable sources. Sulforaphane, or analogue thereof, may be extracted directly from the source material or from extracts prepared from the natural source material. In other embodiments sulforaphane and/or sulforaphane analogues may be synthetic (i.e. man-made).

In embodiments, the mixture of the present disclosure is for use in the treatment or prevention of a disease or condition associated with oxidative stress.

In this context, ‘associated with’ means that oxidative stress may be linked directly or indirectly to the disease or condition. Preferably, the disease or condition is selected from at least one of: cancer, cardiovascular disease, neurological disease, respiratory disease, rheumatoid arthritis, liver disease, kidney disease, inflammation, diabetes, metabolism disorders, or aging.

In embodiments, the mixture of the present disclosure is formulated for administration to a subject in need. The subject may be human or a non-human animal. The mixture may be formulated for administration simultaneously-that is, where components of the mixture are administered to a subject in need at the same time. Simultaneous administration includes pre-mixing components of the mixture, or administering separated components simultaneously. Alternatively components of the mixture may be administered sequentially. To this extent, the present disclosure also encompasses methods of treating or preventing a disease or condition in a subject in need comprising administration of mixtures or compositions as disclosed herein to the subject.

A mixture, components combined to achieve a mixture, or a composition of the disclosure may include liquid preparations for administration by any orifice or surface, including oral, nasal, anal, vaginal, peroral, intragastric, and mucosal (e.g., perlinqual, alveolar, gingival, olfactory or respiratory mucosa) administration. A liquid may include suspensions, syrups or elixirs; and, preparations for parenteral, subcutaneous, intradermal, intramuscular, intraperitoneal or intravenous administration (e.g., injectable administration), such as sterile suspensions or emulsions. In embodiments, liquid mixtures (or components thereof) or compositions may include a carrier, diluent, or excipient such as sterile water, physiological saline, glucose or the like. In embodiments, a mixture (or components thereof) or composition of the disclosure may contain auxiliary substances such as wetting or emulsifying agents, pH buffering agents, gelling or viscosity enhancing additives, preservatives, flavoring agents, colors, and the like, depending upon the route of administration and the preparation desired. Standard texts, such as “REMINGTON'S PHARMACEUTICAL SCIENCE”, 17th edition, 1985, incorporated herein by reference, may be consulted to prepare suitable preparations, without undue experimentation.

In embodiments, a mixture (or components combined to achieve a mixture) or a composition of the disclosure may be provided in the “solid” form of pills, tablets, capsules, caplets and the like, including “solid” preparations which are time-released or which have a liquid tilling, e.g. gelatin covered liquid, whereby the gelatin is dissolved in the stomach for delivery to the gut. If nasal or respiratory (mucosal) administration is desired, compositions may be in a form and dispensed by a squeeze spray dispenser, pump dispenser or aerosol dispenser. Aerosols are usually under pressure by means of a hydrocarbon. Pump dispensers can preferably dispense a metered dose or a dose having a particular particle size.

Liquid preparations may be a preferred form of administration by injection or orally, to animals, children, particularly small children, and others who may have difficulty swallowing a pill, tablet, capsule or the like, or in multi-dose situations. Viscous preparations, on the other hand, may be formulated within the appropriate viscosity range to provide longer contact periods with mucosa, such as the lining of the stomach or nasal mucosa.

The choice of suitable carriers and other additives will depend on the exact route of administration and the nature of the particular dosage form, e.g., liquid dosage form (i.e. a solution, a suspension, gel or another liquid form), or solid dosage form (i.e. pill, tablet, capsule, caplet, time release form or liquid-filled form).

Solutions, suspensions and gels, normally contain a major amount of water (preferably purified water) in addition to the active ingredient. Minor amounts of other ingredients such as pH adjusters (e.g., a base such as NaOH), emulsifiers or dispersing agents, buffering agents, preservatives, wetting agents, jelling agents, (e.g., methylcellulose), colors and/or flavors may also be present. The compositions can be isotonic, i.e., it can have the same osmotic pressure as blood and lacrimal fluid.

The desired isotonicity of the compositions of this disclosure may be accomplished using sodium tartrate, propylene glycol or other inorganic or organic solutes. Sodium chloride is preferred particularly for buffers containing sodium ions.

Viscosity of the compositions may be maintained at the selected level using a pharmaceutically acceptable thickening agent. Methylcellulose is preferred because it is readily and economically available and is easy to work with. Other suitable thickening agents include, for example, xanthan gum, carboxymethyl cellulose, hydroxypropyl cellulose, carbomer, and the like. The preferred concentration of the thickener will depend upon the agent selected. The important point is to use an amount that will achieve the selected viscosity. Viscous compositions are normally prepared from solutions by the addition of such thickening agents.

A pharmaceutically acceptable preservative can be employed to increase the shelf life of the compositions. Benzyl alcohol may be suitable, although a variety of preservatives including, for example, parabens, thimerosal, chlorobutanol, or benzalkonium chloride may also be employed. A suitable concentration of the preservative will be from about 0.02% to about 2% based on the total weight although there may be appreciable variation depending upon the agent selected.

In other embodiments, the mixture of the present disclosure may comprise sulforaphane, or analogue thereof, and glycine, or analogue thereof in a molar ratio of between about 1:1000 to about 1000:1, preferably between about 1:800 to about 800:1, most preferably about 1:500.

In embodiments, components for the preparation of mixtures, or compositions of the present disclosures may comprise compounds, substances or ingredients in amounts between about 1 μmol/kg body weight to about 100 mmol/kg body weight, preferably about 10 μmol/kg body weight to about 1 mmol/kg body weight, more preferably about 100 μmol/kg body weight to about 500 μmol/kg body weight.

In embodiments, sulforaphane, or analogue thereof, may be formulated for administration in a dose of between about 50 nmol/kg body weight to about 500 μmol/kg body weight, preferably about 500 nmol/kg body weight to about 100 μmol/kg body weight, more preferably about 100 nmol/kg body weight to about 50 μmol/kg body weight, more preferably about 50 nmol/kg body weight to about 5 μmol/kg body weight. Preferably, administration is by the oral route.

In embodiments, glycine, or analogue thereof, may be formulated for administration in a dose of between about 10 μmol/kg body weight to about 500 mmol/kg body weight, preferably 10 μmol/kg body weight to 100 mmol/kg body weight, more preferably 10 μmol/kg body weight to 10 mmol/kg body weight, more preferably 50 μmol/kg body weight to 2 mmol/kg body weight.

In another aspect, the present disclosure provides a composition comprising:
sulforaphane, or analogue thereof,
glycine, or analogue thereof,
and optionally at least one ingredient.

In embodiments, the at least one ingredient is selected from the group consisting of: lipids, proteins, carbohydrates, pre-biotics, pro-biotics, essential fatty acids, nucleotides, nucleosides, vitamins, and minerals.

In embodiments, proteins may include, caseins, alpha-lactalbumin, lactoferrin, serum albumin, whey, soy protein, rice protein, corn protein, oat protein, barley protein, wheat protein, rye protein, pea protein, egg protein, sunflower seed protein, potato protein, fish protein, meat protein, immunoglobins and, combinations thereof.

In embodiments, carbohydrates may include lactose, saccharose, maltodextrin, starch, and mixtures thereof

In embodiments, lipids may include: palm olein, high oleic sunflower oil, high oleic safflower oil, canola oil, fish oil, coconut oil, bovine milk fat, or mixtures thereof.

In embodiments, essential fatty acids may include: linoleic acid (LA), α-linolenic acid (ALA) and polyunsaturated fatty acids (PUFAs). The nutritional compositions of the invention may further contain gangliosides monosialoganglioside-3 (GM3) and disialogangliosides 3 (GD3), phospholipids such as sphingomyelin, phospholipids phosphatidylcholine, phosphatidylethanolamine, phosphatidylinositol, phosphatidylserine, and combinations of the foregoing.

In embodiments, prebiotics may include: oligosaccharides optionally containing fructose, galactose, mannose; dietary fibers, in particular soluble fibers, soy fibers; inulin; or mixtures thereof. Preferred prebiotics are fructo-oligosaccharides (FOS), galacto-oligosaccharides (GOS), isomalto-oligosaccharides (IMO), xylo-oligosaccharides (XOS), arabino-xylo oligosaccharides (AXOS), mannan-oligosaccharides (MOS), oligosaccharides of soy, glycosylsucrose (GS), lactosucrose (LS), lactulose (LA), palatinose-oligosaccharides (PAO), malto-oligosaccharides, gums and/or hydrolysates thereof, pectins and/or hydrolysates thereof, and combinations of the foregoing.

In embodiments, probiotics may include: Bifidobacterium, Lactobacillus, Lactococcus, Enterococcus, Streptococcus, Kluyveromyces, Saccharoymces, Candida, in particular selected from the group consisting of Bifidobacterium longum, Bifidobacterium lactis, Bifidobacterium animalis, Bifidobacterium breve, Bifidobacterium infantis, Bifidobacterium adolescentis, Lactobacillus acidophilus, Lactobacillus casei, Lactobacillus paracasei, Lactobacillus salivarius, Lactobacillus lactis, Lactobacillus rhamnosus, Lactobacillus johnsonii, Lactobacillus plantarum, Lactobacillus salivarius, Lactococcus lactis, Enterococcus faecium, Saccharomyces cerevisiae, Saccharomyces boulardii or mixtures thereof, preferably selected from the group consisting of Bifidobacterium longum NCC3001 (ATCC BAA-999), Bifidobacterium longum NCC2705 (CNCM I-2618), Bifidobacterium longum NCC490 (CNCM I-2170), Bifidobacterium lactis NCC2818 (CNCM I-3446), Bifidobacterium breve strain A, Lactobacillus paracasei NCC2461 (CNCM I-2116), Lactobacillus johnsonii NCC533 (CNCM I-1225), Lactobacillus rhamnosus GG (ATCC53103), Lactobacillus rhamnosus NCC4007 (CGMCC 1.3724), Enterococcus faecium SF 68 (NCC2768; NCIMB10415), and mixtures thereof.

In embodiments, nucleotides may include: cytidine monophosphate (CMP) uridine monophosphate (UMP), adenosine monophosphate (AMP), guanosine monophosphate (GMP) or any mixtures or derivatives thereof.

In embodiments, vitamins and minerals include: vitamin A, vitamin B1, vitamin B2, vitamin B6, vitamin Bi2, vitamin E. vitamin K. vitamin C, vitamin D, folic acid, inositol, niacin biotin, pantothenic acid, choline, calcium, phosphorous, iodine, iron, magnesium, copper, zinc, manganese, chloride, potassium, sodium, selenium, chromium, molybdenum, taurine, and L-carnitine, and mixtures thereof. Minerals are usually added in salt form.

In another aspect, the disclosure provides a method of manufacturing a mixtures (or components thereof) and compositions. In embodiments, the method of manufacturing components for use in preparing mixtures and compositions may comprise synthetic production, or extracting, isolating or purifying substances, compounds or ingredients from natural sources, and optionally formulating such substances, compounds or ingredients for administration. Substances, compounds or ingredients may be in liquid or solid form, and can include auxiliary substances and the like.

In embodiments, the method of manufacturing compositions of the present disclosure comprises admixing substances, compounds or ingredients. In embodiments, the method comprises admixing the sulforaphane, or analogue thereof, with the at least one substance and optionally the at least one additional ingredient. The method may optionally further comprise admixing a pharmaceutically acceptable carrier or excipient.

Those skilled in the art will understand that they can freely combine all features of the present disclosure disclosed herein. Further, features described for different embodiments of the present disclosure may be combined.

Furthermore, where known equivalents exist to specific features, such equivalents are incorporated as if specifically referred in this specification.

Further examples of the disclosure are described below. However, it should be noted that the disclosure should not be limited to these examples, and that the aspects and embodiments disclosed herein are susceptible to variations, modifications and/or additions other than those specifically described, and it is to be understood that the disclosure includes all such variations, modifications and/or additions which fall within the scope of the claims.

### EXAMPLES

### Example 1

### Reduction in Oxidative Stress in Zebrafish

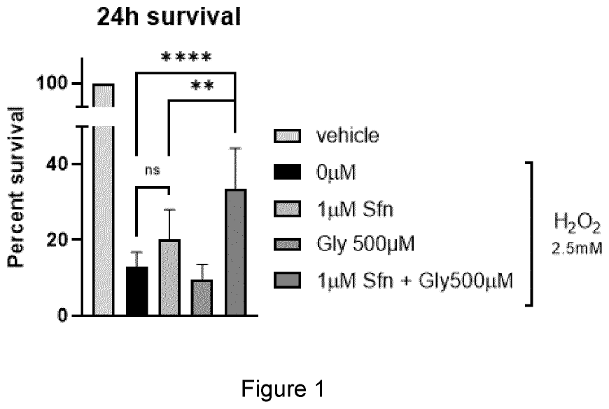

Zebrafish larvae provide a suitable vertebrate model for studying the oxidative stress in vitro³⁵. FIG. 1 shows data produced by examining Zebra fish larvae survival in vitro. Survival assays were performed according to a protocol described previously³⁶ with some modifications. Briefly, wild-type zebrafish larvae

(AB strain, ZFIN ID: ZDB-GENO-960809-7) were used. At 3.5 days post fertilization (dpf), 24 larvae per condition were placed in a 60-mm petri dish with 5 ml of egg water (0.6 g/L Ocean sea salt, Tecniplast). Compounds of interest were directly added into the egg water for 12 h. Subsequently, compounds were removed at 4 days post fertilization (dpf4) and larvae were transferred to a 96-well plate in a volume of 200 μl fresh egg water containing 2.5 mM hydrogen peroxide (H₂O₂; Sigma Merck). Survival was monitored after 24 h of treatment.

Larvae in egg water (vehicle) without additives were compared to various groups in egg water incubated with H₂O₂, namely: vehicle+2.5 mM H₂O₂, vehicle+2.5 mM H₂O₂+1 μM sulforaphane (Sfn; Molport MolPort-003-850-350, stock prepared in DMSO, Sigma Merck), vehicle+2.5 mM H₂O₂+500 μM L-glycine (Gly; Sigma Merck, stock prepared in water), or vehicle+2.5 mM H₂O₂+1 82 M sulforaphane+500 μM L-glycine.

FIG. 1 shows that 100% of larvae in vehicle only survived after 24 h. By comparison, only ˜10% of larvae in vehicle+2.5 mM H₂O₂ survived, which was equivalent to larvae in either vehicle+2.5 mM H₂O₂+1 μM Sfn or vehicle+2.5 mM H₂O₂+500 μM L-glycine. Surprisingly, larvae incubated in vehicle+2.5 mM H₂O₂+1 μM sulforaphane+500 μM L-glycine showed enhanced survival which was statistically significant over the other H₂O₂ groups. Statistical significance was calculated by 2 way ANOVA, Tukey's multiple comparison test, as summarized in Table 1.

TABLE 1
Statistical analysis of data presented in FIG. 1

| | Predicted | 95.00% | | | |
|---|---|---|---|---|---|
| | (LS) mean | Cl of | Below | | Adjusted |
| Groups | diff | diff. | threshold? | Summary | P Value |
| 0 μM vs. 1 μM | −7.077 | −16.06 to | No | ns | 0.1542 |
| Sfn | | 1.907 | | | |
| 0 μM vs. | −20.69 | −29.67 to | Yes | **** | <0.0001 |
| 1 μM Sfn + Gly | | −11.70 | | | |
| 500 μM | | | | | |
| 1 μM Sfn vs. | −13.61 | −24.25 to | Yes | ** | 0.0077 |
| 1 μM Sfn + Gly | | −2.973 | | | |
| 500 μM | | | | | |

ns = not significant,
**** = p < 0.0001?
** = p < 0.01

While illustrative aspects and embodiments have been illustrated and described, including the best mode known to the inventors, those skilled in the art will recognize that variations on the aspects and embodiments are regarded as within the ambit of the disclosure.

### BIBLIOGRAPHY

1. Valko M., Izakovic M., Mazur M., Rhodes C. J., Telser J. Role of oxygen radicals in DNA damage and cancer incidence. Molecular and Cellular Biochemistry. 2004;266:37-56.
2. Valko M., Leibfritz D., Moncola J., Cronin M. D., Mazur M., Telser J. Free radicals and antioxidants in normal physiological functions and human disease. The International Journal of Biochemistry & Cell Biology. 2007;39:44-84. doi: 10.1016/j.biocel.2006.07.001.
3. Jennifer N. Moloney, Thomas G. Cotter. ROS signalling in the biology of cancer. Seminars in Cell & Developmental Biology. Volume 80, 2018, Pages 50-64, https://doi.org/10.1016/j.semcdb.2017.05.023.
4. Bahorun T., Soobrattee M. A., Luximon-Ramma V., Aruoma O. I. Free radicals and antioxidants in cardiovascular health and disease. Internet Journal of Medical Update. 2006; 1:1-17.
5. Chatterjee M., Saluja R., Kanneganti S., Chinta S., Dikshit M. Biochemical and molecular evaluation of neutrophil NOS in spontaneously hypertensive rats. Cellular and Molecular Biology. 2007;53:84-93.
6. Ceriello A. Possible role of oxidative stress in the pathogenesis of hypertension. Diabetes Care. 2008;31 (Supplement 2): S181-S184. doi: 10.2337/dc08-s245.
7. Halliwell B. Role of free radicals in neurodegenerative diseases: therapeutic implications for antioxidant treatment. Drugs & Aging. 2001; 18:685-716.
8. Singh R. P., Sharad S., Kapur S. Free radicals and oxidative stress in neurodegenerative diseases: relevance of dietary antioxidants. Journal, Indian Academy of Clinical Medicine. 2004;5:218-225.
9. Christen Y. Oxidative stress and Alzheimer disease. The American Journal of Clinical Nutrition. 2000;71: 621S-629S.
10. Caramori G., Papi A. Oxidants and asthma. Thorax. 2004;59:170-173.
11. Guo R. F., Ward P. A. Role of oxidants in lung injury during sepsis. Antioxidants & Redox Signaling. 2007;9:1991-2002. doi: 10.1089/ars.2007.1785.
12. Hoshino Y., Mishima M. Antioxidants & redox signaling redox-based therapeutics for lung diseases. Antioxidants & Redox Signaling. 2008;10:701-704. doi: 10.1089/ars.2007.1961.
13. Walston J., Xue Q., Semba R. D., et al. Serum antioxidants, inflammation, and total mortality in older women. American Journal of Epidemiology. 2006; 163:18-26. doi: 10.1093/aje/kwj007.
14 Mahajan A., Tandon V. R. Antioxidants and rheumatoid arthritis. Journal of Indian Rheumatology Association. 2004; 12:139-142.
15 Ze Chen, Ruifeng Tian, Zhigang She, Jingjing Cai, and Hongliang Li. Role of oxidative stress in the pathogenesis of nonalcoholic fatty liver disease Free Radical Biology and Medicine. Volume 152, 2020, Pages 116-141, https://doi.org/10.1016/j.freeradbiomed.2020.02.025
16. Galle J. Oxidative stress in chronic renal failure. Nephrology, Dialysis, Transplantation. 2001;16:2135-2142.
17. Willcox J. K., Ash S. L., Catignani G. L. Antioxidants and prevention of chronic disease. Critical Reviews in Food Science and Nutrition. 2004;44:275-295. doi: 10.1080/10408690490468489.
18. Pizzino G., Bitto A., Interdonato M., et al. Oxidative stress and DNA repair and detoxification gene expression in adolescents exposed to heavy metals living in the Milazzo-Valle del Mela area (Sicily, Italy) Redox Biology. 2014;2:686-693. doi: 10.1016/j.redox.2014.05.003.
19. Jérôme Lugrin, Nathalie Rosenblatt-Velin, Roumen Parapanov and Lucas Liaudet. The role of oxidative stress during inflammatory processes. Biological Chemistry, vol. 395, no. 2, 2014, pp. 203-230.
20. H. Ha, K.H. Kim. Role of oxidative stress in the development of diabetic nephropathy. Kidney Int Suppl, 51 (51) (1995), pp. S18-S21.
21. P. Sankar, S. Subhashree, S. Sudharani. Effect of Trigonella foenum-graecum seed powder on the antioxidant levels of high fat diet and low dose streptozotocin induced type II diabetic rats. Eur Rev Med Pharmacol Sci, 16 (Suppl 3) (2012), pp. 10-17.
22. Houstis N, Rosen ED, Lander ES. Reactive oxygen species have a causal role in multiple forms of insulin resistance. Nature. 440:944-948.
23. Hoehn KL, Salmon AB, Hohnen-Behrens C, et al. Insulin resistance is a cellular antioxidant defense mechanism. Proc Natl Acad Sci U S A 2009; 106:17787-17792.
24. Rosa Vona, Lucrezia Gambardella, Camilla Cittadini, Elisabetta Straface, and Donatella Pietraforte. Biomarkers of Oxidative Stress in Metabolic Syndrome and Associated Diseases. Oxidative Medicine and Cellular Longevity. Volume 2019, Article ID 8267234, https://doi.org/10.1155/2019/8267234
25. Hércules Rezende Freitas, OrcID, Gustavo Da Costa Ferreira, Isis Hara Trevenzoli, Karen De Jesus Oliveira and Ricardo Augusto De Melo Reis. Fatty Acids, Antioxidants and Physical Activity in Brain Aging. Nutrients 2017, 9 (11), 1263; https://doi.org/10.3390/nu9111263.
26. Jiao Luo, Kevin Mills, Saskia le Cessie, Raymond Noordam, Diana van Heemst. Ageing, age-related diseases and oxidative stress: What to do next? Ageing Research Reviews. Volume 57, 2020, 100982 https://doi.org/10.1016/j.arr.2019.
27. Fahey JW, Zhang Y, Talalay P. Broccoli sprouts: an exceptionally rich source of inducers of enzymes that protect against chemical carcinogens. Proc Natl Acad Sci U S A. 1997;94:10367-72.
28. Theresa A. Shapiro, Jed W. Fahey, Albena T. Dinkova-Kostova, W. David Holtzclaw, Katherine K. Stephenson, Kristina L. Wade, Lingxiang Ye &Paul Talalay. Safety, Tolerance, and Metabolism of Broccoli Sprout Glucosinolates and Isothiocyanates: A Clinical Phase I Study. Nutrition and Cancer, 55:1, 53-62, DOI: 10.1207/s15327914nc5501_7.
29. Dinkova-Kostova AT, Kostov RV: Glucosinolates and isothiocyanates in health and disease. Trends Mol Med 2012; 18:337-347
30. Shapiro TA, Fahey JW, Wade KL, Stephenson KK, Talalay P: Chemoprotective glucosinolates and isothiocyanates of broccoli sprouts: Metabolism and excretion in humans. Cancer Epidemiol Biomarkers Prev 2001;10:501-508.
31. Conaway CC, Getahun SM, Liebes LL, et al.: Disposition of glucosinolates and sulforaphane in humans after ingestion of steamed and fresh broccoli. Nutr Cancer 2000;38:168-178.
32. Guerrero-Beltran CE, Calderon-Oliver M, Pedraza-Chaverri J, Chirino YI: Protective effect of sulforaphane against oxidative stress: Recent advances. Exp Toxicol Pathol 2012;64:503-508.
33. Zhang Y, Talalay P, Cho CG, Posner GH. A major inducer of anticarcinogenic protective enzymes from broccoli: isolation and elucidation of structure. Proc Natl Acad Sci USA. 1992;89:2399-2403.
34. R. K. Thimmulappa, K. H. Mai, S. Srisuma, T. W. Kensler, M. Yamamoto, and S. Biswal, “Identification of Nrf2-regulated genes induced by the chemopreventive agent sulforaphane by oligonucleotide microarray,” Cancer Research, vol. 62, no. 18, pp. 5196-5203, 2002.
35. Fang L, Miller YI. Emerging applications for zebrafish as a model organism to study oxidative mechanisms and their roles in inflammation and vascular accumulation of oxidized lipids. Free Radic Biol Med. 2012;53:1411-1420.
36. Endo Y, Muraki K, Fuse Y, Kobayashi M. Evaluation of Antioxidant Activity of Spice-Derived Phytochemicals Using Zebrafish. International Journal of Molecular Sciences. 2020; 21 (3): 1109. https://doi.org/10.3390/ijms21031109.

1. A mixture comprising sulforaphane, or analogue thereof, and glycine, or analogue thereof.

2. The mixture of claim 1, wherein the analogue of sulforaphane is selected from the group consisting of:
6-isothiocyanato-2-hexanone,
exo-2-acetyl-6-isothiocyanatonorbornane,
exo-2-isothiocyanato-6-methylsulfonylnorbornane,
6-isothiocyanato-2-hexanol,
1-isothiocyanato-4-dimethylphosphonylbutane,
exo-2-(1′-hydroxyethyl)-5-isothiocyanatonorborane,
exo-2-acetyl-5-isothiocyanoatonorbomane,
1-isothiocyanato-5-methylsulfonylpentane,
cis-or trans-3-(methylsulfonyl) cyclohexylmethylisothiocyanate, and
combinations thereof.

3. The mixture of claim 1, wherein the mixture comprises sulforaphane and glycine.

4. A method for use in the treatment or prevention of a disease or condition associated with oxidative stress comprising administering a composition comprising sulforaphane, or analogue thereof, and glycine, or analogue thereof to a subject in need of same.

5. The method of claim 1, wherein the mixture is for use in the treatment or prevention of at least one disease or condition selected from the group consisting of:
cancer,
cardiovascular disease,
neurological disease,
respiratory disease,
rheumatoid arthritis,
liver disease,
kidney disease,
inflammation,
diabetes,
metabolism disorders, and
aging.

6. The method of claim 1, wherein sulforaphane, or analogue thereof, and glycine, or analogue thereof are formulated for administration to a subject in need simultaneously or sequentially.

7. The mixture of claim 1, wherein the molar ratio of sulforaphane, or analogue thereof, to glycine, or analogue thereof is between about 1:1000 to about 1000:1.

8. The mixture of claim 1 consisting of:
sulforaphane, or analogue thereof,
glycine, or analogue thereof, and
a pharmaceutically acceptable carrier.

9. The mixture of claim 1, wherein the mixture further comprises at least one ingredient.

10. The mixture of claim 9, wherein the at least one ingredient is selected from the group consisting of:
lipids,
proteins,
carbohydrates,
pre-biotics,
pro-biotics,
essential fatty acids,
nucleotides,
nucleosides,
vitamins, and
minerals.

11. The mixture of claim 1, wherein the mixture is formulated for administration intravenously, subcutaneously or orally.

12. The method of claim 1, wherein the combination of sulforaphane, or analogue thereof, and glycine, or analogue thereof is capable of acting synergistically to treat or prevent a disease or condition when compared to sulforaphane, or analogue thereof, or to glycine, or analogue thereof, when administered separately.

13. A composition comprising:
sulforaphane, or analogue thereof,
glycine, or analogue thereof,
and at least one additional ingredient.

14. The composition of claim 13, wherein the at least one ingredient is selected from the group consisting of:
lipids,
proteins,
carbohydrates,
pre-biotics,
pro-biotics,
essential fatty acids,
nucleotides,
nucleosides,
vitamins, and
minerals.

15. (canceled)
